(19) United States
(12) Patent Application Publication
(10) Pub. No.: US 2025/0386803 A1
(43) Pub. Date: Dec. 25, 2025

(54) SYSTEMS AND METHODS FOR PAIRING DOMESTIC COMPANION ANIMALS

(71) Applicant: Ancestry.com DNA, LLC, Lehi, UT (US)

(72) Inventors: Caitlyn Elizabeth Bruns, Saratoga Springs, UT (US); Ross Eugene Curtis, Cedar Hills, UT (US); Phillip Brooks, San Francisco, CA (US); Robert Hart, Lehi, UT (US); Scott Lewis, Herriman, UT (US); Jenna Morgan Lang, Elk Grove, CA (US)

(21) Appl. No.: 18/752,118

(22) Filed: Jun. 24, 2024

(51) Int. Cl. A01K 29/00; G06F 16/9537; G06F 16/9538; G16B 20/00

(52) U.S. Cl. CPC A01K 29/005; G06F 16/9537; G06F 16/9538; G16B 20/00

(57) ABSTRACT

A computing device may receive an inheritance dataset of a target domestic companion animal that belongs to a first owner, the first owner being a user of an online system. The computing device accesses inheritance datasets of reference panel animals. The reference panel animals are organized into breeds. The computing device compares the inheritance dataset of the target domestic companion animal to the inheritance datasets of the reference panel animals to identify breeds of the target domestic companion animal. The computing device identifies a plurality of matched domestic companion animals in the breeds of the target domestic companion animal. The computing device filters the matched domestic companion animals based on geographical proximity. The computing device causes to display a filtered matched domestic companion animal to the first owner of the target domestic companion animal to indicate potential social matches for the target domestic companion animal.

### FIELD

The disclosed embodiments relate to matching domestic companion animals.

### BACKGROUND

Pet owners often face numerous challenges regarding their pets' socialization and exercise needs. Limited open, pet-friendly spaces in densely populated cities may restrict pets, particularly dogs, from ample exercise and socialization essential for their physical health and emotional well-being. Dogs are inherently social creatures, and a lack of appropriate socialization opportunities may lead to behavioral problems. Establishing connections with other dog owners may be difficult due to the fast-paced, fragmented nature of urban living. Furthermore, pet owners may want to find suitable playmates for their pet based on size and/or energy level of their breed type or dominant breed type for mixed breeds. These challenges and others highlight the need for innovative solutions to improve pet ownership experiences.

### SUMMARY

The system disclosed herein relates to example embodiments that pair domestic companion animals. The system receives an inheritance dataset of a target domestic companion animal that belongs to a first owner, the first owner being a user of an online system. The system accesses inheritance datasets of reference panel animals, wherein the reference panel animals are organized into breeds. The system compares the inheritance dataset of the target domestic companion animal to the inheritance datasets of the reference panel animals to identify one or more breeds of the target domestic companion animal based on the inheritance dataset. The system identifies a plurality of matched domestic companion animals in the one or more breeds of the target domestic companion animal. The system filters the matched domestic companion animals based on a geographical proximity of the other owners compared to a location of the first owner. The system causes to display, at a graphical user interface of a platform maintained by the online system, a filtered matched domestic companion animal to the first owner of the target domestic companion animal to indicate that the filtered matched domestic companion animal and the target domestic companion animal are a potential social match.

In yet another embodiment, a non-transitory computer-readable medium that is configured to store instructions is described. The instructions, when executed by one or more processors, cause the one or more processors to perform a process that includes steps described in the above computer-implemented methods or described in any embodiments of this disclosure. In yet another embodiment, a system may include one or more processors and a storage medium that is configured to store instructions. The instructions, when executed by one or more processors, cause the one or more processors to perform a process that includes steps described in the above computer-implemented methods or described in any embodiments of this disclosure.

### BRIEF DESCRIPTION OF THE DRAWINGS

FIG. 1 illustrates a diagram of a system environment of an example computing system, in accordance with some embodiments.

FIG. 2 is a block diagram of an architecture of an example computing system, in accordance with some embodiments.

FIG. 3 is a flowchart depicting a process that pair domestic companion animals, in accordance with an embodiment.

FIG. 4 depicts a process for matching domestic companion animals, in accordance with an embodiment.

FIG. 5 is a block diagram illustrating example computer architecture, in accordance with an embodiment.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

The figures (FIGS.) and the following description relate to preferred embodiments by way of illustration only. One of skill in the art may recognize alternative embodiments of the structures and methods disclosed herein as viable alternatives that may be employed without departing from the principles of what is disclosed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Configuration Overview

Embodiments of methods, systems, and computer-program products for matching domestic companion animals are provided in the present disclosure. In some embodiments, a computing device may receive a inheritance dataset of a target domestic companion animal that belongs to a first owner, the first owner being a user of an online system. The computing device accesses inheritance datasets of reference panel animals, wherein the reference panel animals are organized into breeds. The computing device compares the inheritance dataset of the target domestic companion animal to the inheritance datasets of the reference panel animals to identify one or more breeds of the target domestic companion animal based on the inheritance dataset. The computing device identifies a plurality of matched domestic companion animals in the one or more breeds of the target domestic companion animal. The computing device filters the matched domestic companion animals based on a geographical proximity of the other owners compared to a location of the first owner. The computing device causes to display, at a graphical user interface of a platform maintained by the online system, a filtered matched domestic companion animal to the first owner of the target domestic companion animal to indicate that the filtered matched domestic companion animal and the target domestic companion animal are a potential social match.

### Example System Environment

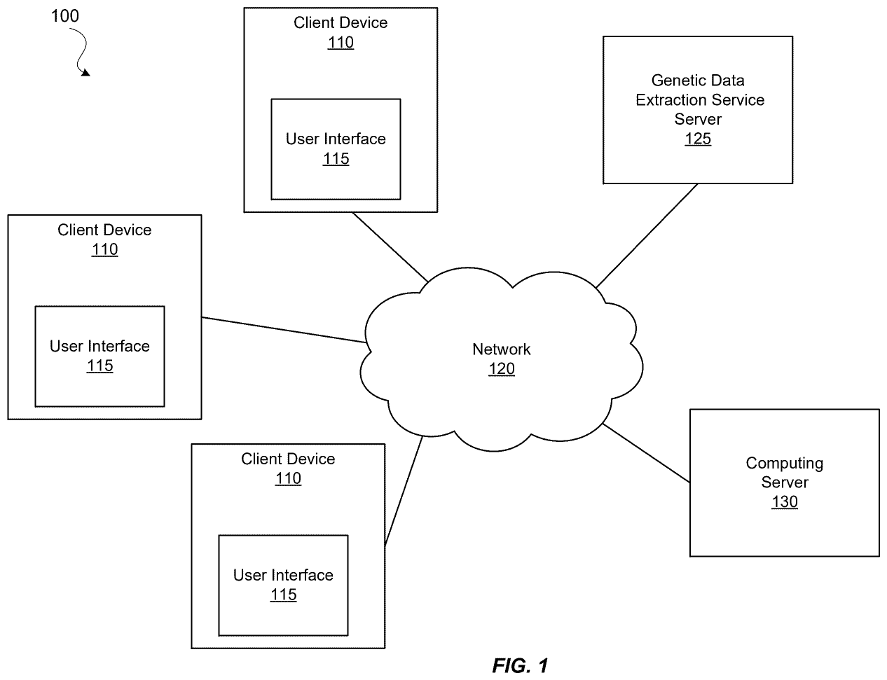

FIG. 1 illustrates a diagram of a system environment 100 of an example computing server 130, in accordance with some embodiments. The system environment 100 shown in FIG. 1 includes one or more client devices 110, a network 120, a genetic data extraction service server 125, and a computing server 130. In various embodiments, the system environment 100 may include fewer or additional components. The system environment 100 may also include different components.

The client devices 110 are one or more computing devices capable of receiving user input as well as transmitting and/or receiving data via a network 120. Example computing devices include desktop computers, laptop computers, personal digital assistants (PDAs), smartphones, tablets, wearable electronic devices (e.g., smartwatches), smart household appliances (e.g., smart televisions, smart speakers, smart home hubs), Internet of Things (IoT) devices or other suitable electronic devices. A client device 110 communicates to other components via the network 120. Users may be customers of the computing server 130 or any individuals who access the system of the computing server 130, such as an online website or a mobile application. In some embodiments, a client device 110 executes an application that launches a graphical user interface (GUI) for a user of the client device 110 to interact with the computing server 130. The GUI may be an example of a user interface 115. A client device 110 may also execute a web browser application to enable interactions between the client device 110 and the computing server 130 via the network 120. In another embodiment, the user interface 115 may take the form of a software application published by the computing server 130 and installed on the user device 110. In yet another embodiment, a client device 110 interacts with the computing server 130 through an application programming interface (API) running on a native operating system of the client device 110, such as IOS or ANDROID.

The network 120 provides connections to the components of the system environment 100 through one or more sub-networks, which may include any combination of local area and/or wide area networks, using both wired and/or wireless communication systems. In some embodiments, a network 120 uses standard communications technologies and/or protocols. For example, a network 120 may include communication links using technologies such as Ethernet, 802.11, worldwide interoperability for microwave access (WiMAX), 3G, 4G, Long Term Evolution (LTE), 5G, code division multiple access (CDMA), digital subscriber line (DSL), etc. Examples of network protocols used for communicating via the network 120 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over a network 120 may be represented using any suitable format, such as hypertext markup language (HTML) or extensible markup language (XML). In some embodiments, all or some of the communication links of a network 120 may be encrypted using any suitable technique or techniques such as secure sockets layer (SSL), transport layer security (TLS), virtual private networks (VPNs), Internet Protocol security (IPsec), etc. The network 120 also includes links and packet-switching networks such as the Internet.

Individuals, who may be customers of a company operating the computing server 130, may provide biological samples of their domestic companion animals for analysis of their genetic data. Individuals may also be referred to as users. In some embodiments, an individual uses a sample collection kit to provide a biological sample (e.g., saliva, blood, hair, tissue) of her domestic companion animal from which genetic data is extracted and determined according to nucleotide processing techniques such as microarray, amplification and/or sequencing. Microarray may include immobilizing probe DNA sequences, onto a solid surface such as a glass slide. Target DNA samples, labeled with fluorescent tags, are then applied to the microarray surface. Through complementary base pairing, the labeled DNA binds to its corresponding probe on the microarray. By detecting the fluorescence emitted by the labeled DNA, genetic data may be extracted. Other probe-based nucleotide identification techniques may also be used. Amplification may include using polymerase chain reaction (PCR) to amplify segments of nucleotide samples. Sequencing may include sequencing of deoxyribonucleic acid (DNA) sequencing, ribonucleic acid (RNA) sequencing, etc. Suitable sequencing techniques may include Sanger sequencing and massively parallel sequencing such as various next-generation sequencing (NGS) techniques including whole genome sequencing, pyrosequencing, sequencing by synthesis, sequencing by ligation, and ion semiconductor sequencing. In some embodiments, a set of SNPs (e.g., 300,000) that are shared between different array platforms may be obtained as genetic data. Genetic data extraction service server 125 receives animal biological samples from users of the computing server 130. The genetic data extraction service server 125 extracts genetic data from the samples and the data may take the form of a set of SNPs. The genetic data extraction service server 125 generates the genetic data of the animals based on sequencing or microarray results. The genetic data may include data generated from DNA or RNA and may include base pairs from coding and/or noncoding regions of DNA.

The genetic data may take different forms and include information regarding various biomarkers of an animal. For example, in some embodiments, the genetic data may be the base pair sequence of an animal. The base pair sequence may include the whole genome or a part of the genome such as certain genetic loci of interest. In another embodiment, the genetic data extraction service server 125 may determine genotypes from DNA identification results, for example by identifying genotype values of SNPs present within the DNA. The results in this example may include a sequence of genotypes corresponding to various SNP sites. A SNP site may also be referred to as a SNP loci. A genetic locus is a segment of a genetic sequence. A locus may be a single site or a longer stretch. The segment may be a single base long or multiple bases long.

The computing server 130 performs various analyses of the genetic data, genealogy data, and users' survey responses to generate results regarding the phenotypes and genealogy of animals of users of computing server 130. Depending on the embodiments, the computing server 130 may also be referred to as an online server, a personal genetic service server, a genealogy server, a breed tree building server, and/or a social networking system. The computing server 130 receives genetic data from the genetic data extraction service server 125 and stores the genetic data in the data store of the computing server 130. The computing server 130 may analyze the data to generate results regarding the genetics or genealogy of animals of users. The results regarding the genetics or genealogy of animals of users may include the breeds of the animals of users, paternal and maternal genetic analysis, identification or suggestion of potential animal playmates, breed information, analyses of DNA data, potential or identified traits such as phenotypes of animals (e.g., diseases, appearance traits, other genetic characteristics, and other non-genetic characteristics including social characteristics), etc. The computing server 130 may present or cause the user interface 115 to present the results to the users through a GUI displayed on the client device 110. The results may include graphical elements, textual information, data, charts, and other elements such as family trees.

In some embodiments, the computing server 130 also allows various users to create one or more genealogical profiles of their animals. For example, a genealogical profile of an animal may be a detailed report including information about an individual animal's ancestry or genealogy. It may include data about the animal's parents, grandparents, and other ancestors, much like a family tree for humans. This profile may be useful in animal breeding and zoological studies, as it allows breeders and researchers to track the lineage of a particular animal, thereby assisting in monitoring inherited traits, maintaining the integrity of a breed, and preventing unwanted genetic conditions. For example, a breeder who wants to maintain certain desirable traits in puppies may create a genealogical profile for a stud to understand what traits may be passed on to their progeny. The user interface 115 controlled by or in communication with the computing server 130 may display the animal's ancestry in a list or as a family tree such as in the form of a pedigree chart. In some embodiments, subject to the user's privacy setting and authorization, the computing server 130 may allow information generated from the animal's inheritance dataset to be linked to the user profile and to one or more of the family trees. The users may also authorize the computing server 130 to analyze their animal's inheritance dataset and allow their animals' profiles to be discovered by other users.

### Example Computing Server Architecture

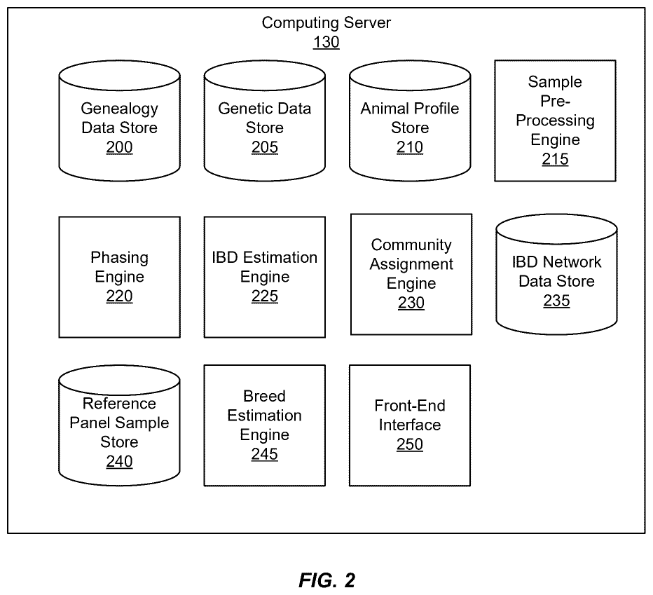

FIG. 2 is a block diagram of the architecture of an example computing server 130, in accordance with some embodiments. In the embodiment shown in FIG. 2, the computing server 130 includes a genealogy data store 200, a genetic data store 205, an animal profile store 210, a sample pre-processing engine 215, a phasing engine 220, an identity by descent (IBD) estimation engine 225, a community assignment engine 230, an IBD network data store 235, a reference panel sample store 240, a breed estimation engine 245, and a front-end interface 250. The functions of the computing server 130 may be distributed among the elements in a different manner than described. In various embodiments, the computing server 130 may include different components and fewer or additional components. Each of the various data stores may be a single storage device, a server controlling multiple storage devices, or a distributed network that is accessible through multiple nodes (e.g., a cloud storage system).

The computing server 130 stores various data of different animals, including genetic data, genealogy data, and survey response data. The computing server 130 processes the genetic data of animals to identify shared identity-by-descent (IBD) segments between animals. The computing server 130 also processes the genetic data of an animal to identify the breed makeup of the animal. The genealogy data and survey response data may be part of animal profile data. The amount and type of profile data stored for each animal may vary based on the information about an animal, which is provided by the user as she creates an account and profile at a system operated by the computing server 130 and continues to build her animal's profile, family tree, and social network at the system and to link her animal's profile with her animal genetic data. Users may provide data via the user interface 115 of a client device 110. Initially and as a user continues to build her animal's genealogical profile, the user may be prompted to answer questions related to the basic information about the user's animal (e.g., name, date of birth, birthplace, etc.) and later on more advanced questions that may be useful for obtaining additional genealogy data. The computing server 130 may also include survey questions regarding various traits of the users' animals such as the animals' phenotypes, characteristics, preferences, habits, lifestyle, environment, etc.

Animal genealogy data may be stored in the genealogy data store 200 and may include various types of data that are related to tracing the lineage or breed of animals. Examples of genealogy data include Animal Identification Numbers, gender, birth locations, date of birth, date of death, sire and dam information, breed information, kinships, breed history, dates and places for significant life events (e.g., birth and death), and the like. In some instances, breed history may take the form of a pedigree of an animal (e.g., the recorded relationships in the breed). The breed tree information associated with an animal may include one or more specified nodes. Each node in the breed tree represents the animal, an ancestor of the animal who may have passed down genetic material to the animal, and the animal's other relatives including siblings, or other offspring of one or more parents in some cases. Genealogy data may also include connections and relationships among different animals based on their genealogical data. The information related to the connections between an animal and its lineage that may be associated with a breed tree may also be referred to as pedigree data or breed tree data.

In addition to user-input data, animal genealogy data may also take other forms that are obtained from various sources such as registries, animal data collectors, and public records. Examples include birth records from breeders, ownership records, death records, veterinary records, pedigree records, migration records, etc. Likewise, genealogy data may include data from one or more lineages or breeds of an animal, pedigree databases, a registry death index, global animal pedigree systems, birth certificate databases, death certificate databases, adoption databases, stud book databases, a vet records database, a migration records database, a property marking database, database of animal-related census, a database of registered owners, a business registration database related to breeders, and the like.

Furthermore, the genealogy data store 200 may also include relationship information inferred from the genetic data stored in the genetic data store 205 and information received from the owners or caretakers of the animals. For example, the relationship information may indicate which animals are genetically related, how they are related, how many generations back they share common ancestors, lengths and locations of IBD segments shared, variants carried by the animal, and the like.

The computing server 130 maintains inheritance datasets of animals in the genetic data store 205. An inheritance dataset of an animal may be a digital dataset of nucleotide data (e.g., SNP data) and corresponding metadata. For example, an inheritance dataset may be genetic data extracted by the genetic data extraction service server 125. An inheritance dataset may contain data the whole or portions of an animal's genome. The genetic data store 205 may store a pointer to a location associated with the genealogy data store 200 associated with the animal. An inheritance dataset may take different forms. In some embodiments, an inheritance dataset may take the form of a base pair sequence of the sequencing or microarray result of an animal. A base pair sequence dataset may include the whole genome of the animal (e.g., obtained from a whole-genome sequencing) or some parts of the genome (e.g., genetic loci of interest). Microarray data may take the form of SNP data at target positions in the genome.

In another embodiment, an inheritance dataset may take the form of sequences of genetic markers in animals. Examples of such genetic markers may include target SNP sites (e.g., allele sites) filtered from the DNA identification results of an animal's genome. A SNP site that is a single base pair long may also be referred to as a SNP locus. A SNP site may be associated with a unique identifier. The inheritance dataset may be in the form of diploid data that includes a sequence of genotypes, such as genotypes at the target SNP site, or the whole base pair sequence that includes genotypes at known SNP sites and other base pair sites that are not commonly associated with known SNPs. The diploid dataset may be referred to as a genotype dataset or a genotype sequence for an animal. A genotype may have different meanings in various contexts. In one context, an animal's genotype may refer to a collection of diploid alleles of a particular animal. In other contexts, a genotype may be a pair of alleles present on two chromosomes for an animal at a given genetic marker such as a SNP site.

Genotype data for a SNP site in an animal's genetic profile may include a pair of alleles. The pair of alleles may be homozygous (e.g., A-A or G-G) or heterozygous (e.g., A-T, C-T). Instead of storing the actual nucleotides, the genetic data store 205 may store genetic data that are converted to bits for animals. For a given SNP site in an animal's genome, frequently only two nucleotide alleles (instead of all 4) are observed. As such, a 2-bit number may represent a SNP site in an animal's genetic data. For example, 00 may represent homozygous first alleles, 11 may represent homozygous second alleles, and 01 or 10 may represent heterozygous alleles. A separate library may store what nucleotide corresponds to the first allele and what nucleotide corresponds to the second allele at a given SNP site within an animal's genome.

A diploid dataset for an animal may also be phased into two sets of haploid data, one corresponding to a sire (father) side and another corresponding to a dam (mother) side. The phased datasets may be referred to as haplotype datasets or haplotype sequences in the context of animals. Similar to genotype, haplotype may have a different meaning in various contexts. In one context, a haplotype for an animal may also refer to a collection of alleles that corresponds to a genetic segment. In other contexts, a haplotype may refer to a specific allele at a SNP site within an animal's genome. For example, a sequence of haplotypes may refer to a sequence of alleles in an animal's genome that are inherited from a parent.

The animal profile store 210 stores profiles and related metadata associated with various animals recorded in the computing server 130. The computing server 130 may use unique animal identifiers to identify different animals and others that may appear in other data sources such as ancestors or historical animals that appear in any pedigree or genealogy database. A unique animal identifier may be a hash of certain identification information of an animal, such as a registered breed name, date of birth, location of birth, known progeny, or any suitable combination of the information. The profile data related to an animal may be stored as metadata associated with an animal's profile. For example, the unique animal identifier and the metadata may be stored as a key-value pair using the unique animal identifier as a key.

An animal's profile data may include various kinds of information related to the animal. The metadata about the animal may include one or more pointers associating inheritance datasets such as genotype and phased haplotype data of the animal that are saved in the genetic data store 205. The metadata about the animal may also include information related to pedigree datasets that include the animal. The profile data may further include declarative information about the animal that was authorized by the owner or caretaker to be shared and may also include information inferred by the computing server 130. Other examples of information stored in an animal profile may include biographic, demographic, and other types of descriptive information such as breed, age, sex, known medical conditions, behavior traits, lineage and the like. In some embodiments, the animal profile data may also include one or more photos of the animal and photos of relatives (e.g., ancestors) of the animal that are uploaded by the owners or caretakers. An owner or caretaker may authorize the computing server 130 to analyze one or more photos to extract information, such as the animal's appearance traits (e.g., color patterns, distinct physical traits, etc.), from the photos. The appearance traits and other information extracted from the photos may also be saved in the profile store. In some cases, the computing server may allow owners or caretakers to upload many different photos of the animals, their relatives, and even companions. Animal profile data may also be obtained from other suitable sources, including pedigree records, veterinary records, rescue organization records, breeder-provided records, photographs, other records indicating one or more traits, and other suitable recorded data.

For example, the computing server 130 may present various survey questions to the owners or caretakers of the animals from time to time. The responses to the survey questions may be stored at the animal profile store 210. The survey questions may be related to various aspects of the animals and the animals' lineage. Some survey questions may be related to animals' phenotypes, while other questions may be related to the environmental factors surrounding the animals.

Survey questions may relate to health or disease-related phenotypes in animals, such as questions related to the presence or absence of genetic diseases or disorders, inheritable diseases or disorders, or other common diseases or disorders that have a breed history as one of the risk factors. Questions regarding any diagnosis of increased risk of any diseases or disorders, and queries concerning wellness-related issues such as a breed history of obesity, common causes of death, etc., may also be included. The diseases identified by the survey questions may be related to single-gene diseases or disorders that are caused by a single-nucleotide variant, an insertion, or a deletion in the animals. The diseases identified by the survey questions may also be multifactorial inheritance disorders in animals that may be caused by a combination of environmental factors and genes. The computing server 130 may obtain data on an animal's disease-related phenotypes from survey questions about the health history of the animal and its breed, and also from health records uploaded by the owner or veterinarian.

Survey questions also may be related to other types of phenotypes such as appearance traits of the animals. A survey regarding appearance traits and characteristics may include questions related to fur color, eye color, ear shape, tail length, paw size, size and type of horns in certain species, feather color in birds, scale pattern in reptiles, and so on. A survey regarding other traits also may include questions related to animals' sensory abilities such as sight, smell, hearing or taste abilities. A survey regarding traits may further include questions related to animals' health conditions such as lactose tolerance in mammals, certain disease resistances, performance ability, responses to certain medications, and so on. Other survey questions regarding an animal's physiological traits may include dietary traits, sensory traits such as the ability to sense certain scents or respond to certain stimuli. Traits may also be collected from pedigree records, veterinary records and breeder-provided records.

The computing server 130 also may provide various survey questions related to the environmental factors of animals. In this context, an environmental factor may be a factor that is not directly connected to the genetics of the animals. Environmental factors may include animals' living conditions, routines, and training habits. For example, a survey regarding animals' living conditions may include questions related to types of habitat, whether an animal is kept indoors or outdoors, type and frequency of social interactions, access to spaces for exercise, etc. Other questions may be related to the animals' diet such as preference for certain types of food, known allergies, frequency of feeding, and dietary supplements. A survey related to routines and behaviors may include questions regarding daily activity levels, grooming behaviors, sleeping habits, responses to training, and behavioral quirks. Additional environmental factors may include diet amount (calories, macronutrients), physical abilities (e.g., agility, endurance), family type (single pet household or multi-pet household), and aspects of care provision (type of veterinary care, frequency of check-ups, preventive healthcare measures).

In addition to storing the collected survey data in the individual animal profile store 210, the computing server 130 may store certain responses corresponding to genealogical and genetic data specifically in the animal genealogy data store 200 and animal genetic data store 205. This separation may allow for a focused analysis in respective domains, helping to trace lineage, detect inherited traits, and understand genetic predispositions within the species or individual animal. Consolidating these data in dedicated stores may provide insights into an animal's genetic and genealogical makeup, further enriching the knowledge base to ensure optimal animal care, selective breeding decisions, or conservation strategies.

In some instances, the animal profile store 210 may be a large-scale data store. The animal profile store 210 may, for instance, include at least 10,000 data records in the form of animal profiles, each linked to one or more genetic data sets and one or more genealogical data entries. In other embodiments, the data records related to animal profiles may be significantly higher. They may range from 50,000 records, or even to as high as 100,000, 500,000, 1,000,000, 2,000,000, 5,000,000, and 10,000,000 data records for very extensive data collections. In each of these cases, each animal profile would be paired with one or more genetic data sets and one or more genealogical entries. This vast collection of data presents an immense resource for comprehending the genetic makeup, lineage, health predispositions and environmental influences on diverse animal species, thereby contributing enormously to veterinary medicine, wildlife conservation, and other animal-relevant disciplines.

The sample pre-processing engine 215 may receive and pre-process data received from various sources to transform the data into a format utilized by the computing server 130. For genealogy data, the sample pre-processing engine 215 may receive data from an individual via the user interface 115 of the client device 110. To collate the data (e.g., genealogical data and survey data related to animals), the computing server 130 may provide an interactive user interface on the client device 110 to display interface elements, whereby users may provide animal genealogy data and survey data. Additional data may be gleaned from the digital scans of public records or veterinary records. The data may be manually provided or automatically extracted e.g., via optical character recognition, performed on records from animal registries, rescue centers, wildlife census records, relevant governmental records, or any other piece of printed or online material. Some records may be obtained by digitizing written records such as older veterinary records, birth or hatching records, death records, etc.

The sample pre-processing engine 215 may also receive raw data from the genetic data extraction service server 125, which may conduct laboratory analysis of biological samples from animals. This server may generate DNA identification results in the form of digital data which is then received by the sample pre-processing engine 215. Mutations that get passed down to offspring are commonly linked to single-nucleotide polymorphisms which are substitutions of a single nucleotide that occur in a precise position in the genome. The sample pre-processing engine 215 may transform this raw base pair sequence into a sequence of genotypes of target SNP sites facilitating identification of these genetic diversity markers within an animal's genetic data set. Samples may involve varying numbers of SNP sites, such as at least 10,000, 100,000, 300,000, or even 1,000,000 SNP sites. After conversion, the identified SNP sites may be provided to the phasing engine 220. This engine processes an animal's diploid genotypes to generate a pair of haplotypes (one set from each parent), providing a detailed foundation for genetic inquiry into lineage, inherited attributes, or species-specific traits.

The phasing engine 220 may process a diploid inheritance dataset into a pair of haploid inheritance datasets for an animal, and may also perform imputation of SNP values at certain sites where alleles are missing. An animal's haplotype may refer to a collection of alleles, representing a sequence that is inherited from a single parent. This process ensures a more detailed understanding of an animal's unique genetic character and inheritance patterns, thereby supporting genetic studies and related concerns ranging from understanding disease predispositions to conservation genetics.

Phasing in an animal context may include the process of determining the assignment of alleles (specifically heterozygous alleles) to particular chromosomes. Due to conditions inherent in sequencing or microarray and other constraints, a DNA identification result may often include data for a pair of alleles at a specific SNP locus of a pair of chromosomes, but it may fail to discern the specific chromosome each allele belongs to. So, the phasing engine 220 may use a genotype phasing algorithm to assign each allele to its respective chromosome. The genotype phasing algorithm may be constructed based on an assumption of linkage disequilibrium (LD), which suggests that haplotypes, in the form of allele sequences, tend to cluster together. The phasing engine 220 may be programmed to derive phased sequences that are commonly observed in many other animal samples-put differently, haplotype sequences from different animals tend to cluster together. The development of a haplotype-cluster model may help ascertain the probability distribution of a haplotype incorporating a sequence of alleles. This model may be trained using labeled data constituted by known phased haplotypes from a trio, in this context, a parent pair and their offspring. A trio may serve as an effective training sample because the correct phasing of the offspring may be decidedly inferred by comparing the offspring's genotypes with their parents' inheritance datasets. The haplotype-cluster model may be gradually formulated in conjunction with the phasing process involving numerous unphased genotype datasets. It may also be leveraged to impute one or more missing data points.

As an example, the phasing engine 220 may deploy a directed acyclic graph model like a hidden Markov model (HMM) to carry out the phasing of a target genotype dataset for an animal. This directed acyclic graph may comprise multiple levels, each level having multiple nodes that represent different potential haplotype clusters within the animal dataset. An emission probability of a node, representing the probability of encountering a specific haplotype cluster given an observation of the genotypes, may be established based on the probability distribution of the haplotype-cluster model. A transition probability from one node to another may be initially designated a non-zero value, which may be tweaked as the directed acyclic graph model and the haplotype-cluster model undergo training. There may be numerous potential paths in traversing different levels of the directed acyclic graph model. The phasing engine 220 may pinpoint a statistically likely path, such as the most probable path or a path more likely than 95% of other possible paths, based on the transition probabilities and emission probabilities. A fitting dynamic programming algorithm like the Viterbi algorithm may be deployed to determine this path. The determined path may represent the phasing result. U.S. Pat. No. 10,679,729, entitled “Haplotype Phasing Models,” granted on Jun. 9, 2020, describes example embodiments of haplotype phasing.

A phasing algorithm may also produce phasing results that accurately span a large genomic distance and cross-chromosome separation in terms of animal haplotype separation. This attribute may allow for accurate identification and analysis of long genetic linkages and more significant amounts of data per animal, enabling more comprehensive genealogical and genetic studies. For example, in some embodiments, an IBD-phasing algorithm may be used, which is described in further detail in U.S. Patent Application Publication No. US 2021/0034647, entitled “Clustering of Matched Segments to Determine Linkage of Dataset in a Database,” published on Feb. 4, 2021. The computing server 130 may be used to receive a target animal genotype dataset and multiple additional individual genotype datasets that incorporate the haplotypes of other animals. These additional animals may be corresponding reference panels or individuals linked to the target animal (for instance, via a family tree or breed lineage). The computing server 130 may then create multiple sub-cluster pairs of primary and secondary parental groups. Each of these sub-cluster pairs may exist within a window, a concept similar to that used in the breed estimation engine 245 and associated Hidden Markov Model (HMM) disclosures, indicating a specific genomic segment. How windows are precisely divided and defined may be consistent across the Phasing Engine 220 and HMM or vary as needed. Each sub-cluster pair may correlate to a genetic locus. In certain scenarios, the pairs may include a primary parental group with a matched set of haplotype segments chosen from the supplementary individual datasets and a secondary group with another matched set of haplotype segments drawn from these datasets. The computing server 130 may create a super-cluster of a parental side by connecting the primary and secondary parental groups across various genetic loci (across several sub-cluster pairs). The generation of the super-cluster may involve producing a prospective parental side assignment of parental groups across a group of sub-cluster pairs that represent a set of genetic loci in the enumerated genetic loci. The computing server 130 may discern the number of common additional animal genotype datasets that are categorized in the proposed parental side assignment. Based on the number of common additional animal genotype datasets, the computing server 130 may determine the proposed parental side assignment to be a part of the super-cluster. Any suitable algorithms, including heuristic scoring approaches, bipartite graph strategies, or other efficient methods may be employed to generate the super-cluster. Following this process, the computing server 130 may construct a haplotype phasing of the target animal from the super-cluster of the parental side.

The IBD estimation engine 225 may measure the extent of shared genetic segments between a pair of animals, leveraging phased genotype data (such as haplotype datasets) stored in genetic data store 205. IBD sections may be segments identified in a pair of animals, potentially inherited from a shared ancestor. For each animal pair, the IBD estimation engine 225 may pull up two haplotype data sets. It may break each haplotype dataset sequence into several windows with each window containing a set number of SNP (Single Nucleotide Polymorphism) sites (approximately 100 SNP sites, for instance). The IBD estimation engine 225 may zone in on one or more seed windows where all SNP site alleles in at least one of two compared animals' phased haplotypes are identical. It may then expand the match from these seed windows to adjacent windows until it hits the end of a chromosome or discovers a homozygous mismatch, ruling out phasing or imputation errors. The IBD estimation engine 225 may then identify the cumulative length of the matched segments, alternatively known as IBD segments. This length may be gauged in terms of genetic distance in centimorgans (cM), a genetic length unit. Two genomic positions that are one cM apart may have a 1% chance of a recombinatory event in each meiosis between the two positions. Wherever individual pairs have an IBD segment length surpassing a specific threshold (for example, 6 cM), the computing server 130 may store the relevant data in an appropriate data store such as genealogy data store 200. U.S. Pat. No. 10,114,922, entitled “Identifying Ancestral Relationships Using a Continuous stream of Input,” granted on Oct. 30, 2018, and U.S. Pat. No. 10,720,229, entitled “Reducing Error in Predicted Genetic Relationships,” granted on Jul. 21, 2020, describe example embodiments of IBD estimation.

Typically, animals that are closely related tend to share a considerable amount of IBD segments, and these segments may be generally longer, either individually or combined across one or multiple chromosomes. Conversely, animals with a more distant relation tend to share fewer IBD segments, and these segments may be usually shorter in length, whether individually or combined across one or multiple chromosomes. For instance, closely related animals within the same breed or lineage may often share upwards of 71 cM (centimorgans) of IBD, akin to the genetic overlap you may find between third cousins in humans. More distantly related animals may share less than 12 cM of IBD segments. The notion of IBD affinity may be used to indicate the degree of relatedness in terms of IBD segments between two animals. This IBD affinity may be quantified by measuring the length of IBD segments that two animals have in common.

The community assignment engine 230 may allocate individual animals to one or more genetic communities based on their genetic data. In this sense, a genetic community may refer to a specific breed or a group of animals descended from shared ancestry. How finely or broadly these genetic communities are classified may vary, contingent upon the methods used and the specific use case. For instance, in certain scenarios, communities may be as broad as Canines, Felines, Equines, etc. In some embodiments, breeds may be further divided based on geographical breeding history or notable lineage variations such as ‘Labradors bred in North America’, ‘Labradors from championship lineage’, ‘Working German Shepherds’, etc. The community classification may also be impacted by whether a population is purebred or mixed. For mixed breeds, the classification may be further divided based on the combination of different breeds within a geographical region.

The community assignment engine 230 may assign animals to one or more genetic communities based on their inheritance datasets using machine learning models trained by unsupervised learning or supervised learning. In an unsupervised approach, the community assignment engine 230 may generate data representing a partially connected undirected graph. In this approach, the community assignment engine 230 represents animals as nodes. Some nodes may be connected by edges whose weights are based on IBD affinity between two animals represented by the nodes. For example, if the total length of two animals' shared IBD segments does not exceed a predetermined threshold, the nodes may not connected. The edges connecting two nodes may be associated with weights that are measured based on the IBD affinities. The undirected graph may be referred to as an IBD network. The community assignment engine 230 may use clustering techniques such as modularity measurement (e.g., the Louvain method) to classify nodes into different clusters in the IBD network. Each cluster may represent a genetic community. The community assignment engine 230 may also determine sub-clusters, which represent sub-communities. The computing server 130 may save the data representing the IBD network and clusters in the IBD network data store 235. U.S. Pat. No. 10,223,498, entitled “Discovering Population Structure from Patterns of Identity-By-Descent,” granted on Mar. 5, 2019, describes example embodiments of community detection and assignment.

The community assignment engine 230 may also categorize animal genetic communities utilizing supervised techniques. For instance, inheritance datasets from recognized genetic communities (e.g., animals of confirmed breed origins) may serve as labeled training sets for these supervised models. Machine learning classifiers that operate on a supervised basis, such as logistic regressions, support vector machines, random forest classifiers, and neural networks, may be trained using these labeled training sets. A trained classifier may have the capacity to differentiate between two or more classes. A binary classifier may, for example, be trained for every breed of interest to ascertain whether a target animal's inheritance dataset belongs or may not belong to that particular breed. A multi-class classifier, such as a neural network, may also be trained to determine the most probable breed affiliation of the target animal's inheritance dataset from among several possible breeds.

The reference panel sample store 240 may retain reference panel samples for various breeds. A reference panel sample may be the genetic data of an animal that most representatively captures the genetic profile of a specific breed or lineage. The genetic data from animals bearing the typical alleles of a certain breed or lineage may be utilized as reference panel samples. For instance, the computing server 130 may first select for allegedly purebred samples and use the purebred samples to expand the reference panel. For example, the purebred samples may be used for training a variety of machine learning models tasked with classifier roles, such as determining whether a target inheritance dataset aligns with a particular animal breed or lineage, ascertaining the breed composition of a mixed-breed animal, and ascertaining the accuracy of any genetic data analysis. This may be accomplished by calculating a posterior probability of a classification result from a classifier.

The breed estimation engine 245 may estimate the breed composition of a inheritance dataset of a target animal. The inheritance datasets employed by the breed estimation engine 245 may be genotype datasets or haplotype datasets. For example, the breed estimation engine 245 may estimate the breed or lineage origins based on the animal's genotypes or haplotypes at the SNP sites. Consider a simple example of three ancestral populations corresponding to Labrador, German Shepherd, and Beagle breeds. An admixed animal may have non-zero estimated breed proportions for all three ancestral populations, with an estimate such as [0.05, 0.65, 0.30]. This indicates that the animal's genome is 5% attributable to Labrador ancestry, 65% attributable to German Shepherd ancestry, and 30% attributable to Beagle ancestry. The breed estimation engine 245 may formulate the breed composition estimate and store the estimated breeds in a data store of computing server 130, associating it with a particular animal via a pointer.

In some embodiments, the breed estimation engine 245 may divide the target inheritance dataset for an animal into several windows (for example, around 1000 windows). Each window incorporates a small range of SNPs (Single Nucleotide Polymorphisms), such as 300 SNPs. The breed estimation engine 245 may utilize a Directed Acyclic Graph model to determine the breed composition of the target inheritance dataset. This Directed Acyclic Graph may represent a trellis of an Inter-Window Hidden Markov Model (HMM). The graph may involve a sequence of several node groupings. Each node group, indicative of a window, contains numerous nodes. These nodes may represent different possible labels of genetic communities (for example, breeds) for the window. A node may be identified with one or more breed labels. To illustrate, a level may include of a first node with a first label signifying the likelihood that the window of SNP sites corresponds to a specific breed, and a second node with another label representing the probability that the window of SNPs correlates with a second breed. Each level may include multiple nodes so that there are several potential paths to traverse the directed acyclic graph.

The nodes and edges in the directed acyclic graph model may be correlated with various emission probabilities and transition probabilities. An emission probability linked to a node may embody the likelihood that the window pertains to the breed labeling the node, given the observed SNPs in the window. The breed estimation engine 245 may determine the emission probabilities by comparing SNPs in the window corresponding to the target inheritance dataset with equivalent SNPs in the windows within various reference panel samples of different breed communities, which are stored in the reference panel sample store 240. The transition probability between two nodes may represent the chance of transitioning from one node to another across two levels. The breed estimation engine 245 may determine a statically likely path, such as the most probable path, or a likely path that is at least more probable than 95% of other potential paths based on the transition and emission probabilities. To determine the path, a suitable dynamic programming algorithm like the Viterbi algorithm or the forward-backward algorithm may be used. Once the path is decided, the breed estimation engine 245 may determine the breed composition of the target inheritance dataset by discerning the label compositions of the nodes included in the determined path. U.S. Pat. No. 10,558,930, entitled “Local Genetic Ethnicity Determination System,” granted on Feb. 11, 2020, and U.S. Pat. No. 10,692,587, granted on Jun. 23, 2020, entitled “Global Ancestry Determination System” describe different example embodiments of ethnicity estimation.

The front-end interface 250 may display various results and data handled by the computing server 130. For example, these results may include the genetic relationship between an animal and another member of the same species, the group allocation of the animal, its breed prediction, phenotype appraisal, genealogic data search, lineage tree, and related animal profile, etc. The front-end interface 250 may tools for users to manage their animal profile and data trees (e.g., lineage trees). Users may view different public lineage trees saved on the computing server 130, and search for animals and their genealogic information via the front-end interface 250. The computing server 130 may suggest or permit the user to manually check and choose potentially related animals (e.g., offspring, ancestors, nearby kin) to include in the user's data tree. The front-end interface 250 may be a graphical user interface (GUI) providing various details and graphical components. The form of front-end interface 250 may vary. In one case, the front-end interface 250 may be a software application that may be displayed on an electronic device such as a computer or a smartphone. The software application may be developed by the entity controlling the computing server 130 and be downloaded and installed on the client device 110. In another case, the front-end interface 250 may take the form of a webpage interface of the computing server 130 that allows users to access their family tree and genetic analysis results through web browsers. In yet another case, the front-end interface 250 may provide an application program interface (API).

### Example Process for Assigning Individuals to one or more Ethnicities

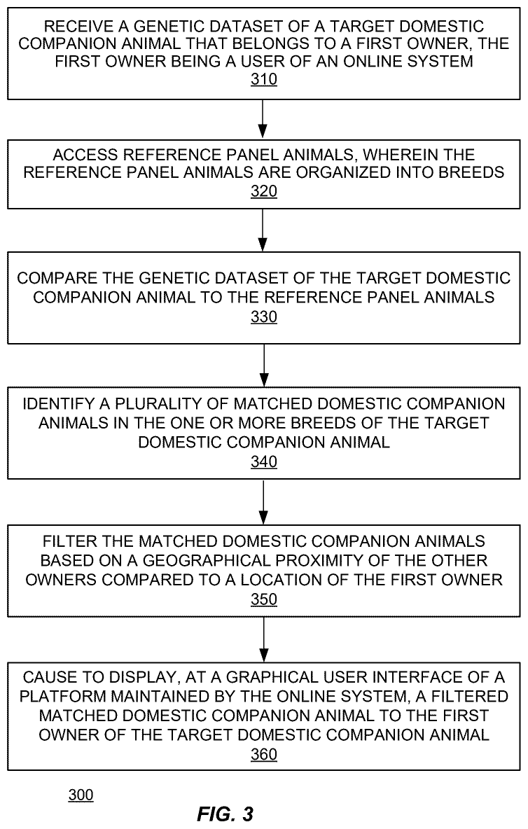

FIG. 3 is a flowchart depicting an example process 300 for pairing domestic companion animals, in accordance with some embodiments. The process may be performed by one or more engines of the computing server 130 illustrated in FIG. 2. The process 300 may be embodied as a software algorithm that may be stored as computer instructions that are executable by one or more processors. The instructions, when executed by the processors, cause the processors to perform various steps in the process 300. In various embodiments, the process may include additional, fewer, or different steps. While various steps in process 300 may be discussed with the use of computing server 130, each step may be performed by a different computing device.

In some embodiments, the process 300 may include receiving an inheritance dataset of a target domestic companion animal that belongs to a first owner (step 310). The inheritance dataset may be any form of genetic dataset that is extracted by the genetic data extraction service server 125 and/or stored in the genetic data store 205. The owner of the target domestic animal may be a user of an online system, such as the computing server 130. A domestic companion animal may refer to a pet, which may be kept for companionship and emotional support. In this disclosure, domestic companion animals may sometimes also be referred to as pets, animals, or non-human mammals. Domestic animals have undergone selective breeding for generations to emphasize traits such as behavior, size, and appearance that make them amenable to cohabitation with humans. In some cases, the animals may provide their owners with emotional comfort and a sense of well-being. As used herein, a domestic companion animal may be a dog, though it should be appreciated that a domestic companion animal may also include, but is not limited to, other companion animals like cats and birds that are often part of a home and closely engaged with their owners.

While in this disclosure matching of domestic companion animals such as pets is used as the primary example of matching animals, in various embodiments the processes 300 and 400 and various engines described in FIG. 2 may be applied to other animals such as matching of non-human animals, non-human mammals, and other non-human vertebrates.

Using the process 300, owners may engage with an online platform to find a suitable match for their pet, indicating a proactive role in seeking social companionship for their pet and/or themselves.

The online system may be a digital (for e.g., Internet-based) platform that is designed to match domestic companion animals based on their inheritance datasets. For example, one of the purposes of the online system may be to identify potential companion matches for a user's pet, based on the breeds of the animals or a more granular breed type. The online system may facilitate potential social matches for domestic companion animals, leading to improved socialization for pets, and fostering a sense of community among users. The online system may be the computing server 130, which may provide genetic and genealogical research service to the human users and additionally provide matching of companion animals among the users. Further features of the computing server 130 is described above in the discussion in associated with FIGS. 1 and 2.

In some embodiments, the owner may use a specially designed swab to collect DNA from their pet. For example, this may be done by rubbing the swab on the inside of the pet's cheek for a specified duration. This process may collect epithelial cells from which DNA may be extracted. The type and design of the swab may ensure that a sufficient quantity and quality of cells are collected while ensuring comfort and safety for the pet. After the swabbing process is complete, the swab may be stored to preserve the integrity of the cells and DNA. This may involve sealing it in a sterile container or package, in some cases facilitated by a chemical buffer to prevent degradation of the genetic material captured within the collection device. A preservation solution may also be used to preserve the DNA contained within the swab. The swab or the preservation solution may then be sent by the owner to a designated lab or facility associated with the online system. This may be via standard postal service or a pre-arranged courier service provided by the system. On reaching the lab, DNA may be extracted from the swab or the preservation solution. For example, microscopic cells collected on the swab may be broken down to release the DNA within them. This DNA may be cleaned and concentrated, making it ready for identification such as by sequencing or microarray. The lab may perform any suitable laboratory techniques such as microarrays or DNA sequencing to determine genetic inheritance of the sample. The inheritance data may take the form of variations in the pet's DNA, represented as genetic markers or single-nucleotide polymorphisms (SNPs). The inheritance dataset for the pet may include the DNA data in a certain suitable format. This dataset may include information on identified genetic markers and, in some cases, may include predicted traits or characteristics of the pet based on the identified markers. The generated inheritance dataset may be stored within the online system's database, associated with the owner's profile and the pet's information. Further details of the collection and generation of the inheritance datasets of the animals is discussed above in association with FIGS. 1 and 2.

In some embodiments, the inheritance dataset of the domestic companion animal may be a dataset stored in the genetic data store 205. In some embodiments, the inheritance dataset of the domestic companion animal may be phased, which means that genomic data is organized according to the chromosomal copies inherited from each parent. Phased data may provide information about which parental haplotype specific genetic variations are originated, allowing for increased accuracy in finding genetic matches of the animals. The phasing engine 220 may use an Identity-by-Descent (IBD) phasing algorithm to identify long-range cross-chromosome phasing of haplotypes. This algorithm helps separate the phasing of a genotype for the majority or almost the entire genome.

Continuing with reference to FIG. 3, in some embodiments, the process 300 may include accessing inheritance datasets of reference panel animals (step 320). The reference panel may be a database containing genetic information on a wide range of animals. The reference panel animals may be organized into different types of datasets such as breeds, breed types, etc.

A breed may refer to a specific group of domestic animals having homogeneous appearance (or phenotype), homogeneous behavior, and/or other characteristics that differentiate it from other organisms of the same species. In case of dogs, a breed often may refer to a lineage that is officially recognized by a relevant kennel authority and adhere to specific criteria in terms of size, color, shape, temperament, and other discernable traits. In some cases, breeds may be established and maintained through controlled, selective breeding processes that emphasize the preservation and propagation of particular desirable traits. Examples of different breeds of dogs include Labrador Retriever, German Shepherd, Bulldog, and Beagle, among others.

A breed type may refer to a more granular classification of animal breeds based on certain shared characteristics of certain breeds. A breed type may include a set of breeds. The categories of breed types may group breeds with similar functions, traits, or origins. With respect to dogs, breed types may include categories such as working dogs, toy breeds, or sporting dogs. This classification may be used not only to identify a dog's breed but also to predict certain behavioral patterns or needs, such as energy level or size, which are factored into finding suitable matches for dogs to play with. Sporting dogs may be bred to assist hunters in locating and retrieving quarry. Examples include Labrador Retrievers, Cocker Spaniels, and Pointers. Herding dogs may be bred to gather, herd, and protect livestock. Border Collies, Australian Shepherds, and Shetland Sheepdogs are some examples. Working dogs may be bred to perform jobs like pulling sleds, guarding property, or performing water rescues. Examples are Boxers, Siberian Huskies, and Saint Bernards. Toy dogs are small companion or lap dogs and may include breeds such as Pomeranians, Chihuahuas, and Pugs.

In some embodiments, the reference panel may serve as a basis for breed determination and compatible pairing by holding inheritance datasets derived from multiple animals representing each breed. For example, each breed in the reference panel may be elucidated with a minimum of number (for e.g., 2, 3, 4, 5, 6, 7, 8, 9, 10, 15, 20, etc.) of distinct, purebred samples. The extensiveness of the panel's breed representation may vary depending on the number of available samples from a particular breed. In some embodiments, the reference panel may include more than 50, 100, 200, 300, or 400 breeds. In some embodiments, each breed represented in the reference panel may include at least five distinct samples obtained from purebred animals of the given reference breed. In some embodiment, a breed may be assigned if there's a confidence level greater than a threshold prediction (for e.g., 75%, 80%, 85%, 90%, 95%, 98%, 99%, 99.5%,etc.) in its identification for that breed.

In some embodiments, to build a reference panel of animals, inheritance data from a wide array of the animal breeds may be collected to form a genetic network. In some cases, some of these samples may be collected through self-identification, where the animal owners provide the breed information. Alternatively, or additionally, the samples may be sourced from academic institutions such as research institutions and universities that have conducted genomic studies on various breeds of the animal. For animals considered purebred and papered (e.g., registered with a kennel club), it may be desired to verify an animal's purebred status and/or that of its ancestral line. For example, it may be desired to confirm that the animals' parents and grandparents all belong to the same breed. In some embodiments, to constitute a breed on the reference panel, one may need to have at least a number of distinct samples of the supposed purebred animal. For example, at least five distinct samples of the purebred animal may be needed. While five may be a minimum number, some more dominant breeds may feature a higher number of samples, leading to a more robust representation. More samples may be needed to distinguish between closely related breeds (e.g., Belgian Sheepdog versus Belgian Tervuren, Alaskan Malamute versus Greenland Sledge Dog) or breed sizes (e.g., Toy Poodle versus Miniature Poodle versus Standard Poodle).

Purebred animals may serve as desirable reference points for drawing out the genetic profiles of specific breeds. While traditional methods of determining a purebred may involve looking at documented pedigrees and breed papers, genetic testing may provide a more objective means of verifying an animal's breed composition. The basis for using genetic testing to identify purebred animals may involve comparing the animal's inheritance dataset with the genetically analyzed samples in a referenced panel. For example, each breed on this panel is represented by several samples of supposed purebred animals. Through this comparison, the presence and proportion of identifiable breed-specific genetic markers in an animal's DNA may be identified. In some embodiments, when there is a strong correlation between an animal's genetic markers and a specific breed represented in the reference panel, that animal may be classified as a purebred of that specific breed. In some embodiments, reference panel may be generated based on the process discussed in the reference panel sample store 240.

In some embodiments, the breed identification process may rely on established benchmarks. For example, a breed may be recognized if a confidence level threshold in its identification is met or exceeded. The confidence level threshold ensures differentiation between similar breeds. For example, the breeds that pass this threshold may end up being reported out. Examples of thresholds may include 75%, 80%, 85%, 90%, 95%, 98%, 99%, or 99.5%. In some embodiments, various thresholds may be applied in a classifier model that is trained for breed prediction. The thresholds may be part of the classifier model to generate an output selection of breeds. In some embodiments, the thresholds may also be used downstream of the classifier model to filter out breeds that have low confidence scores. In some embodiments, a threshold value may be selected based on a balance between having a stricter threshold that reduces errors and a lower threshold that allows the identification of more breeds. In some embodiments, the threshold values may be learned in a machine learning process with a classifier that has an objective function of increasing the accuracy of matching animals with the same breed type. For example, if a threshold is set too high, the number of breeds that are assigned to an animal will reduce and the breed type of the animal may be assigned differently compared to another animal even though both animals may have similar size, color, weight, and/or energy level. If a threshold is set too low, there may be too many breeds assigned to an animal, which may result in an unmanageable or noisy dataset. The determination of the right level of threshold may be based on matching the identification of breeds that are normally recognized by users. For example, users may be readily able to identify one or more breeds that an animal belongs to by observing the animal, the threshold may be select to a level so that the breed identification process will make a similar result. The threshold(s) may additionally or alternatively be determined on the basis of reference panels for particular breeds; for example, thresholds may be lower for reference panels that are particularly small (e.g., for rare breeds) compared to reference panels that are particularly large (e.g., for common breeds).

In some embodiments, in defining a threshold, a breed is assigned if a particular confidence level (e.g., 80%) is achieved or surpassed. For example, if the breed identification process relies on an 80% confidence threshold and the statistical probability that observed genetic characteristics would classify the animal as a given breed meets or exceeds 80%, then the animal would be assigned the identified breed. Thus, the confidence level threshold may play a critical role in ensuring accuracy. One benefit is that it may prevent misclassification of breeds that have similar genetic markers. Implementing a high confidence threshold (e.g., at or above 90%, at or above 95%, at or above 98%, at or above 99%, or at or above 99.5%), can beneficially avoid the breed prediction algorithm from falsely identifying an animal's breed, especially when the genetic markers may be present in more than one breed. Using a confidence threshold may enhances the user's trust in the application. The confidence threshold may enhance the accuracy and reliability of the animal-matching application for its users. In embodiments, the classifier model for breed classification, as described above, is applied on a window-by-window basis, such that the prediction is regarding which breed a particular window corresponds to with sufficient confidence for assignment thereto. Thus, a percentage of a companion animal's breed makeup may be determined by assessing the percentage of windows that are assigned, using the classifier model and confidence metrics discussed above, to respective breeds.

In some embodiments, after the reference panel is built, the computing server 130 may conduct checks to ensure the data's accuracy and reliability. This process may include verifying whether the breeds in the panel accurately represent animals in the larger population. In some embodiments, the computing server 130 may check the defined breeds for consistent performance when identifying an animal's genetic matches.

Continuing with reference to FIG. 3, in some embodiments, the process 300 may include comparing the inheritance dataset of the target domestic companion animal to the inheritance datasets of the reference panel animals (step 330) to determine the breed composition of the target domestic companion animal. This step 330 may result in identifying one or more breeds of the target domestic companion animal based on the inheritance dataset. In some embodiments, the computing server 130 may prepare the inheritance dataset from the target animal and those from the reference panel for comparison. In some embodiments, the computing server 130 may divide the inheritance dataset of the target animal into a plurality of segments that may be referred to as a window. Within each window, the computing server 130 may determine the most likely breed label of the window based on the genetic markers in the window compared to various reference panel samples' genetic markers within the same corresponding window. In some embodiments, the assignment of a breed label may also depend on neighboring windows' assignments because genetic sequences may be inherited in larger segments from the same ancestor. After assigning breed labels in various windows, the computing server 130 may determine the breed composition of the target animal. In various embodiments, other additional genetic analysis techniques may be used to determine the breed labels. For example, in some embodiments, the computing server 130 may apply one or more engines described in FIG. 2, such as the phasing engine 220, the community assignment engine 230 and/or the breed estimation engine 245, to compare the inheritance datasets of animals to determine whether two animals are a genetic match.

In some embodiments, an overall composition of breeds may be generated, such as 20% breed X, 15% breed Y, 2% breed Z, etc. In some cases, a target domestic companion animal may be reported to have many breeds, such as 15 or more breeds. In some embodiments, to reduce the false positive of breed identification, only a breed that exceeds a certain percentage threshold is considered to be included in the breed composition. In some embodiments, the percentages of the breeds may be based on the composition of genomic segments that are best matched to various breeds. For example, if a particular breed only accounts for 2% of the breed composition, the computing server 130 may determine that the breed is below the threshold and not include the breed in the composition. The threshold value may be selected manually or in a machine learning process with the goal of balancing between having a stricter threshold that reduces errors and a lower threshold that allows the identification of more breeds, as discussed above on how various thresholds may be selected.

Continuing with reference to FIG. 3, in some embodiments, the process 300 may include identifying a plurality of matched domestic companion animals in the one or more breeds of the target domestic companion animal (step 340). For example, the breeds (or breed composition) of other domestic companion animals may be determined using a similar way described in step 330 for each domestic companion animal. In turn, the target domestic companion animal may be matched with other domestic companion animals by breeds or by breed types. In some embodiments, top N breeds are first selected for each domestic companion animal, and the top N breeds are used for matching. In some embodiments, after selecting top N breeds, the top N breeds are further categorized into breed types. In turn, the domestic companion animals are matched using breed types other than directly with the breeds.

By way of example, a purebred can be matched with other purebreds of the same breed based on the breed composition determination. In some embodiments, the purebred may also be matched with mixed breed animals whose dominant breed in their breed composition is the same as the purebred. Similarly, a first mixed breed animal whose highest proportion of any breed is 25% of breed X can be matched with a second mixed breed animal whose highest proportion of any breed is also breed X, regardless of the percentage of the breed X in the second animal as long as breed X is the highest proportion in the composition of the second animal. This way of matching can be expanded to match the top two breeds in the estimate, the top three breeds, etc., or can be generalized to the breed type of the primary (and/or secondary) breed.

In some embodiments, the computing server 130 may also determine the matched domestic companion animals based on a breed type of the target domestic companion animal. The breed type of the target domestic companion animal may help in determining suitable matches. With respect to dogs, examples of breed types may include working dogs, toy breeds, sporting dogs, among others. The computing server 130 may identify the breed type of the target companion animal. The computing server 130 may then use this breed type to filter the pool of potential matches. For example, the computing server 130 may search for other animals within its database that share the same breed type as the target animal. Sharing the same breed type implies the animals may have similar characteristics, behaviors, or needs since these breed types often group breeds with similar functionality, origins, or traits. By restricting the selection of matches to the target animal's breed type, the computing server 130 may generate a list of potential matches that not only share similar genetics but are also more likely to have a similar disposition or energy levels. This breed type compatibility may thus heighten the probability of successful social matches.

In some embodiments, the computing server may use the breed type of the target companion animal as a factor in the matching process. The breed type may serve as an indicator in determining suitable matches among potential candidates in the database. As breeds belonging to the same breed type, the animals generally are more compatible socially. With respect to dogs for instance, dogs within the working dogs breed type are often robust, active, and intelligent. Conversely, dogs in the toy breeds category are typically smaller in size and may not require as much physical activity. By taking into account these breed type characteristics, the computing server may propose matches with higher likelihoods of compatibility, both in terms of genetics and behaviors or energy levels. This breed type compatibility may enhance the chances of successful social connections for the target companion animal and improve the overall user experience.

In some embodiments, the computing server 130 may determine the matched domestic companion animals based on two dominant breed types or more. For example, once the two dominant breed types are identified, the computing server uses this information to filter potential matches. It may scan the entire dataset of animals in its system to find those with matching or closely related breed types. Animals of the same or similar breed types will likely exhibit common characteristics and behaviors, making them suitable for social interaction. One desirable advantage of determining matched domestic companion animals based on the two dominant breed types lies in enhanced compatibility and provides efficiency in filtering potential matches. With respect to dogs, different breeds often have distinct behaviors, energy levels, and requirements for social interaction. Identifying and matching based on the two dominant breed types means potentially identifying dogs who share similar traits and behaviors. This strategy enhances the likelihood of compatible playmates, ensuring the interactions between the dogs to be more harmonious and enjoyable.

For example, in a hypothetical scenario of a dog owner, Jenna, has recently adopted a mixed-breed dog, Sam. Sam is primarily a Golden Retriever with traits of an Alsatian, which is determined through the collection and analysis of Sam's DNA data. In determining Sam's two dominant breed types, the computing server identifies the Golden Retriever breed and the Alsatian breed in Sam's genetic data. These are breeds that are generally known for their intelligence, energy level and sociability. When Jenna uses the dog matching application, the computing server may search its dataset and begins to filter potential playmate matches based on these dominant breed types. For example, the computing server may look for other dogs in its dataset that are primarily a combination of Golden Retriever, Alsatian, or breeds that bear similarity in terms of behavior, temperament, and energy levels. Let's assume it identifies Bella, a German Shepherd (closely related to Alsatian) and Labrador mix, and Max, a purebred Golden Retriever, as potential matches. Both Bella and Max share similar breed types to Sam, hence, are likely to have compatible characteristics and behaviors. The computing server may present Bella and Max as suitable playmates for Sam, enhancing the potential for successful and enjoyable interactions between the dogs.

In some embodiments, the computing server 130 may determine the matched domestic companion animals based on an energy level of the target domestic companion animal. One of the factors that may influence a domestic companion animal's suitability as a playmate or companion for another one is its energy level. Domestic companion animals with mismatched energy levels may not interact well, leading to discomfort or even hostility in a worst-case scenario. Therefore, in some embodiments, the computing server 130 may consider the energy level of the target domestic companion animal when determining possible matches. With respect to dogs for example, energy levels may relatively correspond with their breed types. For instance, Border Collies and Labrador Retrievers typically have high energy levels, while breeds like Basset Hounds or Bulldogs are generally more laid back.

By considering the dominant breed types of a dog, which the computing server may identify from the dog's inheritance dataset, the computing server may estimate its energy level. For example, energy levels may be reasonably deduced from their dominant breed types identified from their genetic data. When determining matches, the computing server may consider the estimated energy levels of all the dogs in its database. It then identifies and presents those dogs whose energy levels align with that of the target dog. This strategy may provide that the matched dogs have comparable activity requirements, leading to enjoyable and balanced interactions. Using energy levels as a desirable criterion for matching dogs offers numerous benefits. Compared to dogs with mismatched energy levels, dogs with similar energy levels are likely to play and interact more harmoniously, resulting in a safer and more enjoyable experience. This also helps in reducing potential stress for the dogs and their owners, thus encouraging continued use of the platform for future social matches.

For example, a target dog, Daisy, is a Labrador Retriever, a breed typically known for high energy levels. Using Daisy's inheritance dataset, the computing server identifies her dominant breed type as Labrador Retriever. Since Labradors are generally known for their high energy level, the computing server estimates Daisy's energy level as high. The computing server then compares Daisy's high energy level to the estimated energy level of all other dogs within its database. The computing server may then find high-energy dogs as potential playmates for Daisy. Therefore, the computing server matches Daisy with other high-energy dogs to ensure they may play well together and enjoy their time.

In some embodiments, the computing server 130 may determine the matched domestic companion animals based on a size, weight or color of the target domestic companion animal. In certain embodiments, the computing server may collect the inheritance dataset of the target domestic companion animal alongside additional information like size, weight, and color, provided by the owner through the user interface of the online system. For example, the owner of a golden retriever, Bella, may input on the user interface of the online system that Bella weighs 70 lbs, measures 24 inches in height, and has a golden coat color. Once the computing server receives Bella's data, it conducts a thorough comparison process. It compares Bella's size, weight, and color with the similar parameters associated with other pets within its database. It seeks to identify pets which closely match Bella's attributes and, thus, may be potential companions suited to her specifics. In addition to genetic data, the computing server may consider physical attributes such as size and weight, especially for determining matches that are appropriate for physical play. Matching a large, 70 lbs dog like Bella with a significantly smaller, lighter pet may not result in comfortable interaction. Therefore, the computing server may filter out smaller and lighter pets, focusing on those which closely resemble Bella's size and weight, to assure safe and balanced interactions. In some embodiments, each breed type may be defined based on the average size, weight, or color data of breeds.

Color, such as hair color, may be a relevant feature for certain owner preferences or animal behavioral patterns. Some owners may wish to match their pets with others of similar color. Certain dog breeds may show behavioral tendencies toward dogs of similar colors due to dominance or mating instincts. Incorporating color in the matching process caters to these possibilities, ensuring a comprehensive and tailored pet matching service.

Continuing with reference to FIG. 3, in some embodiments, the process 300 may include filtering the matched domestic companion animals based on a geographical proximity of the other owners compared to a location of the first owner (step 350). For example, the online system may gather the location data from the owner of the target domestic companion animal. The owner may provide the location details during profile setup or directly via location tracking by the online system, given that they authorize this action. Although the precision may vary, the granularity of location information ideally extends down to city level to ensure more relevant matches, though state or country-level inputs are also possible. This location data may be utilized to compute geographical proximity to other users of the system.

The computing server may employ standardized proximity algorithms to calculate distances or match location data based on latitude and longitude coordinates. These proximity calculations may help distinguish which other users (dog owners) are near enough to be potential matches. By using match location data, the computing server may filter the matched domestic companion animals. For example, all potential matches, such as dogs whose genetic data aligns with the target dog, may be sifted through this geographical filter. The end goal is to generate a list of matched dogs whose owners live within an acceptable geographical range relative to the first owner's location. A curated match list may be displayed to the user via a graphical user interface of the online system. This may provide the first owner to review suggested social matches for their dog that are not only genetically compatible but also within feasible physical reach for arranging playdates or meetings. The physical reach feature may be set or changed by the user based on selection criteria for matches (e.g., coat color, etc.). For example, some users may want to find any compatible playmate for their target dog(s) based on compatible breed, breed type, etc., but others may be willing to travel longer distances based on a rarer set of features. For example, finding a purebred Harlequin Great Dane may make the “feasible physical reach” much longer than finding a large dog of the same breed type as a Great Dane or having a Great Dane as the most prominent breed of a mixed breed dog. Advantageously, this domestic animal matching process may provide an improved user experience by combining phenotypic and/or genetic compatibility with geographical convenience.

For instance, Ben is a user of an online domestic companion animal matching platform. Ben lives in San Francisco, California, and he has a Siberian Husky named Max. Ben is interested in finding potential playmates for Max. Upon registering to the platform, Ben uses a specially designed swab to collect DNA from Max and sends the swab to the platform by mail. The platform uses the swab to generate genetic data for Max. Using Max's genetic data, the platform may identify Max's breed. The platform then moves to find matches for Max, such as other Siberian Huskies with compatible phenotypic and/or genetic traits. For this example, let's assume that the platform identifies 100 potential companions for Max across the United States. However, not all these matches are practically suitable due to the geographical distance. Here's where the geographical proximity filter becomes desirable. The platform may check the location data provided by the owners of the matched dogs. After applying this filter, the platform may find out that 15 Huskies among these 100 matches are located within San Francisco, specifically within a range of 20 miles from Ben's registered location.

Hence, by applying the proximity filter, the platform narrows down the list to these 15 Huskies, presenting a more realistic and actionable list of potential companions for Max. Ben may now comfortably set playdates for Max, with the confidence that the suggested matches are both genetically compatible with his pet and conveniently located for arranging meetings. Genetic compatibility may be determined based on various processes discussed in this disclosure. In some embodiments, the two animals' inheritance datasets may each be analyzed to determine their respective breed composition using the process described at step 330. In turn, genetic compatibility may be determined based on the degree of overlap of breeds between the two animals. In some embodiments, any processes that are described in FIG. 2 associated with comparing two inheritance datasets may be used to define genetic compatibility.

Genetic compatibility may be based on a suitable degree of overlap; for instance, a compatible match shares the first two most prominent breeds of a mixed breed dog, but those two most prominent breeds only account for 25% of the total number of identified breed types. This potential match may be determined to have a lower degree of “genetic compatibility” than matching a purebred Golden Retriever with a Goldendoodle. In embodiments, suitable modalities, including heuristics, machine-trained methods, express user preferences, or combinations thereof, may be utilized to assess genetic compatibility for and between particular companion animals. For example, a user may express that they only desire for playdates with animals of a particular size, regardless of breed, or that they desire playdates with animals of a particular breed composition, regardless of other characteristics that may be based on the breed prediction. Such preferences may be utilized in tandem with heuristics and/or other approaches to determine a degree of overlap between the inheritance datasets of two companion animals and their suitability as potential playdate matches. Continuing with reference to FIG. 3, in some embodiments, the process 300 may include causing to display, at a graphical user interface of a platform maintained by the online system, a filtered matched domestic companion animal to the first owner of the target domestic companion animal to indicate that the filtered matched domestic companion animal and the target domestic companion animal are a potential social match (step 360). For example, once the computing server receives the inheritance dataset of a user's animal, it processes this data to generate relevant matches based on genetic compatibility and geographical proximity. The computing server may perform all the computations, filtering, and comparison tasks. Following the completion of data processing and match generation, the computing server may interact with the platform's Graphical User Interface (GUI) to display the results. The GUI is the visual front-end of the platform, through which users interact with the platform. The computing server transmits data to the GUI, converting all the processed information into a user-friendly format.

The data received at the GUI is used to populate the user's dashboard on the platform, showcasing the filtered matches for their animal. Information about these matches may include the breed, age, personality characteristics, owner contact information, and even pictures of the potential companions. The displayed matches may be those animals that are not only genetically compatible but also conveniently located to facilitate easy socialization.

In some embodiments, the platform organizes and presents this information in a way that clearly communicates to the user that these matched animals may be good social matches for their animal. This presentation may be in the form of a list or cards, with an option to “like” or “pass” similar to popular dating apps, or a customizable format that aligns with user preferences. The objective is to make it easy and intuitive for the user to understand the potential social match and to take the next step, whether that's reaching out to other dog owners, setting up playdates, or saving the match for future reference.

In some embodiments, upon generating potential social matches for a user's domestic companion animal, the computing server may provide a messaging system for users to interact. This allows the owner of the target domestic companion animal to initiate a conversation with the other owners of the matched animals, encouraging communication and collaboration. For example, when a user views a potential match, the computing server may send a request to activate the messaging feature associated with that match. This initiates a protocol in the system's server, prompting it to display a message button or link on the GUI next to each potential match's information. When clicked or tapped by the user, a text box, instant message window, or similar messaging interface would appear for the user to send a message to the selected pet owner. The computing server may be programmed to prioritize user privacy and ensure personal contact details are not shared directly. Messages may be sent within the platform's interface and do not disclose any personal contact information of either party unless willingly shared by the users within their conversation. This allows communication between animal owners to take place in a secure manner while maintaining the privacy of all parties involved.

In some embodiments, the messaging feature may be desired for user engagement on the platform. By enabling direct communication, the process of coordinating playdates or social gatherings for their pets is simplified. It fosters a sense of community among users as they share their pets' experiences, swap advice, and build relationships, thereby enhancing the overall user experience. In some embodiments, the computing server 130 may also facilitate animal care services (e.g., dog-sitting) or community establishment (e.g., owners with dogs of breed X that have digestive issues, grooming advice, etc.). Additional alternative embodiments may include the use of breed estimates and genetic compatibility informing a user's decision on whether and which companion animals to obtain. For example, the user may want to confirm that a companion animal will be compatible with an existing companion animal and/or that a companion animal will be compatible with a desired lifestyle (e.g., active users may want a companion animal well-disposed to trail running).

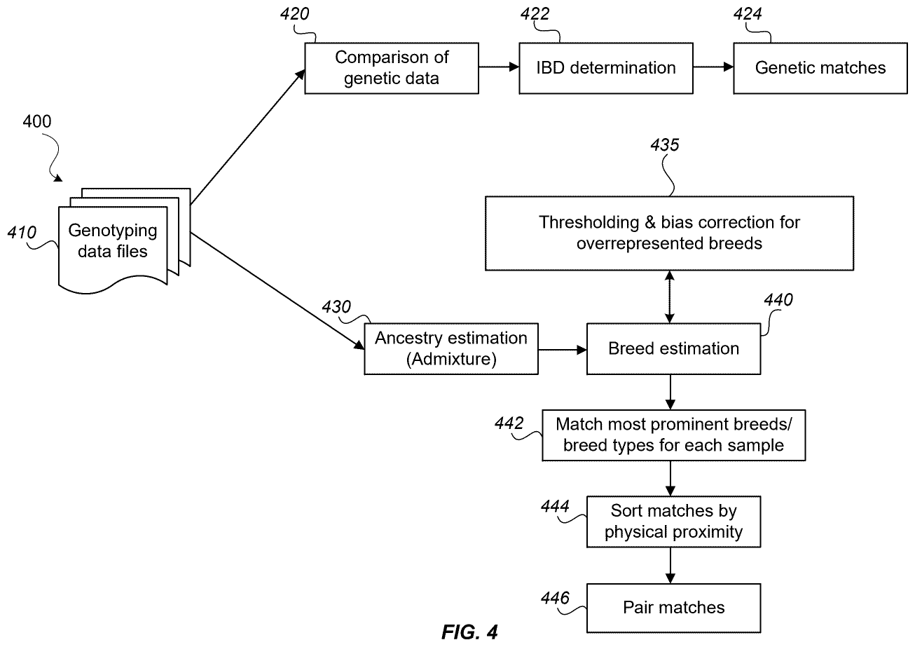

FIG. 4 is a block diagram depicting an example process 400 for pairing domestic companion animals, in accordance with some embodiments. The process may be performed by one or more engines of the computing server 130 illustrated in FIG. 2. The process 400 may be embodied as a software algorithm that may be stored as computer instructions that are executable by one or more processors. The instructions, when executed by the processors, cause the processors to perform various steps in the process 400. In various embodiments, the process may include additional, fewer, or different steps. While various steps in process 400 may be discussed with the use of computing server 130, each step may be performed by a different computing device.

In some embodiments, the process 400 may include genotyping data files such as inheritance datasets of one or more animals (step 410). Genotyping may refer to analyzing the genetic makeup of a domestic animal's DNA. This forms the basis of identifying the breed or breeds that comprise a domestic companion animal, such as a dog. The process begins when a computing server receives data files containing the genetic information of the animal. These files may be obtained through a DNA collection method, such as a swab, and the DNA is then extracted and sequenced to determine the various genetic markers. The computing server may organize this information into a format suitable for comparison. The genotyping 410 of data files may include using one or more of the engines described in FIG. 2, such as the sample pre-processing engine 215 and the phasing engine 220.

Continuing with FIG. 4, the computing server may use this genotyped file to provide two or more types of analyses, such as identifying familial or genetic matches as indicated in route 420 and to identify social matches based on breeds as indicated in route 430.

For the identification of familial or genetic matches, the computing server 130 may compare (step 420) the genetic markers within the data of the target animal with other inheritance datasets stored in the genetic data store 205. In the context of IBD, two or more animals share an IBD segment if they have identical genetic sequences in that segment from a recent common ancestor. During the comparison process, the computing server may identify these IBD segments (step 422). These segments are essentially shared stretches of DNA. The more significant the quantity and length of the IBD segments that the target animal shares with another animal, the more closely related they are. Once the computing server has compared all the genetic markers and identified all the IBD matches, it may compile this information. The result is a list of matches and the degree of relatedness between the target animal and each matched animal, based on the quantity and size of the IBD segments they share (step 424). The identification 422 of IBD segments and determination of genetic matches 424 may be performed by the phasing engine 220 and IBD estimation engine 225. The computing server 130 may report any IBD matches that the server found in the genetic data store 205 to the owner of the target domestic companion animal.

Alternatively, or additionally, the computing server 130 may determine the breed composition of the target domestic companion animal and identify potential social matches for the domestic companion animal, as indicated by route 430. The process 300 is an example of route 430. The computing server 130 may examine the target animal's genetic data in the context of benchmark profiles in reference panels for ancestry estimation. The computing server 130 may identify areas of the target animal's genome that closely match those of the reference breeds. The computing server may determine the percentage of the animal's genome that corresponds to each breed based on the number and size of the matching segments. The computing server may provide an ancestry estimation (step 430), which presents the likely breed composition of the target animal. The breed estimation may be performed by the breed estimation engine 245 or another suitable algorithm that compares the inheritance dataset of the target domestic companion animal to the inheritance datasets of the reference panel animals. This estimation may include the various breeds that contributed to the animal's genetic makeup and the proportions in which they are represented. This ancestry report may provide a comprehensive summary of the target animal's breed composition based on its genetic data.

The computing server may determine or use a threshold such that breed estimates lower than the threshold are not reported (step 435). The breeds estimates may be added to top breeds. For example, the computing server may not report breed estimates below a certain threshold. The thresholding process performed by the computing server may serve as a means to refine and simplify the data presented to users. For example, in analyzing the genetic data of the target animal, the computing server may determine an initial breed composition estimation that may contain several breeds, including those that make up a relatively small proportion of the animal's genetics. The thresholding process may follow this initial estimation. The computer server may aggregate any breed proportions that are below the threshold into the top breeds (those that make up a larger proportion of the pet's genetic makeup), and those smaller proportions are not reported. The aim of this process may be to simplify the breed composition report, preventing an over-complicated multiple-breed result, particularly where the contribution of certain breeds would be negligible.

For example, if the initial breed estimate for a dog showed a 4% Beagle composition, this percentage may not be reported separately due to the threshold. Instead, that 4% would be added to a predominant breed in the dog's genetic makeup, thus reducing the number of breeds reported in the final result. This thresholding process may be coupled with a bias correction for overrepresented breeds to ensure more accuracy. Due to the method of collecting reference data, it may be possible that some popular or common breeds are overrepresented in the reference panel or database. This overrepresentation may potentially skew the breed estimation unintentionally towards these breeds. The bias correction mechanism may be designed to counteract this. It adjusts the breed estimations by reducing the influence of overrepresented breeds, thereby providing a more balanced and accurate depiction of a dog's breed composition.

As the computing server compares the genetic markers of the domestic companion animal to the reference panel, it may identify which breed or breeds the animal most closely resembles (step 440). The computing server may compile this identification into a data file. This file may contain the results of the comparison: a clear breakdown of what breed or breeds contribute to the pet's genetic makeup. The result may then be used for further assessments, such as matching dogs for playdates based on breed, size, and energy level or even identifying potential health concerns associated with certain breeds.

The computing server may match a target animal to a matched animal based on the breeds of those animals (step 442). For example, the breeds (or breed composition) of other domestic companion animals may be determined using the process described in step 330 for each domestic companion animal. In turn, the target domestic companion animal may be matched with other domestic companion animals by breeds or by breed type. In some embodiments, top N breeds are first selected for each domestic companion animal and the top N breeds are used for matching. In some embodiments, after selecting top N breeds, the top N breeds are further categorized into breed types. In turn, the domestic companion animals are matched using breed types other than directly with the breeds.

The computing server may sort matches by physical proximity through the integration of user provided location data and geolocation technology (step 444). For example, when a user registers their animal for matching on the online matching platform, they provide their location data. This may be as broad as a country or state or as specific as a city or town. The computing server may maintain a database where it stores the geographical location of all the animal samples registered in the service. For example, when identifying potential matches for a user's animals, the server filters the matched animals based on their geographical proximity to the user's location. This filtering may be setup to match within certain radiuses, such as within the same city, or within a predetermined distance. After potential matches are filtered based on geography, the computing server may sort these matches by their physical proximity to the user's dog, for example, nearest to furthest.

After a user evaluates potential matches and selects a specific match for potential interaction, the computing server may pair the user's animal to the match (step 444). For example, the server may verify that the selected match is still valid, affirming that the matched dog's owner has not deactivated their account or changed crucial matching data that may affect the compatibility. Once the match is confirmed, the server may proceed to initiate the pair. This process may involve updating both users' accounts to show the matched status and generating a shared interaction space. A feature within the online platform, like a messaging system, may be activated or made accessible for the paired users. This may allow them to communicate within the platform, discuss their dogs' compatibility, and potentially arrange meet-ups or play dates. As the user's interaction progresses, the server may update the match status accordingly. This feature may include marking a match as ‘successful’, ‘pending’, or ‘declined’ based on user feedback or communication activity.

### Computing Machine Architecture

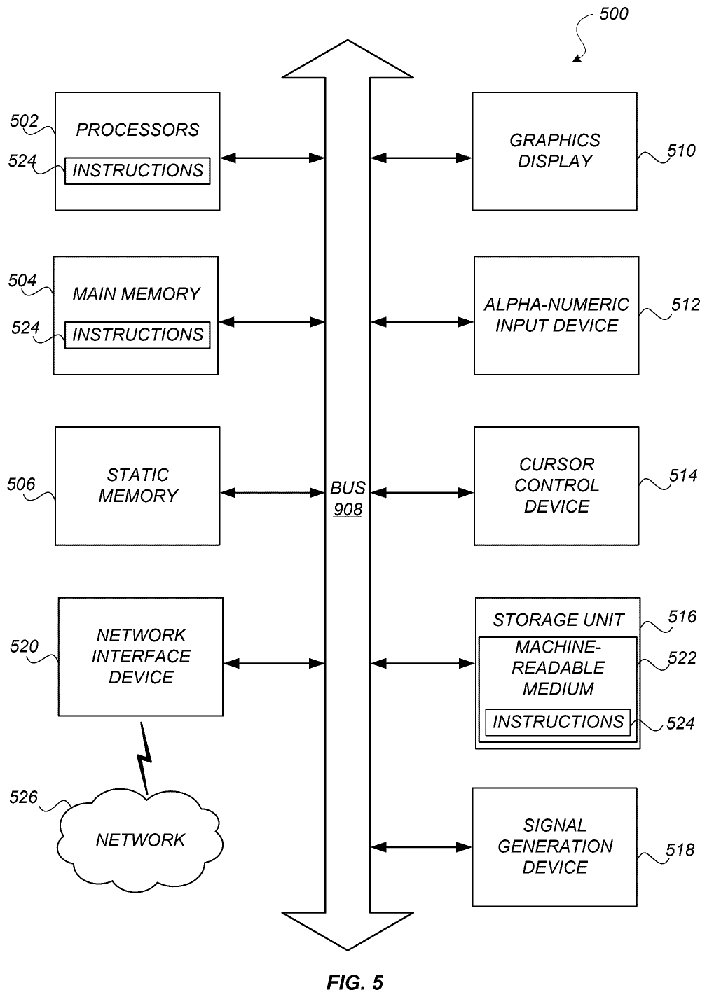

FIG. 5 is a block diagram illustrating components of an example computing machine that is capable of reading instructions from a computer-readable medium and executing them in a processor (or controller). A computer described herein may include a single computing machine shown in FIG. 5, a virtual machine, a distributed computing system that includes multiple nodes of computing machines shown in FIG. 5, or any other suitable arrangement of computing devices.

By way of example, FIG. 5 shows a diagrammatic representation of a computing machine in the example form of a computer system 500 within which instructions 524 (e.g., software, source code, program code, expanded code, object code, assembly code, or machine code), which may be stored in a computer-readable medium for causing the machine to perform any one or more of the processes discussed herein may be executed. In some embodiments, the computing machine operates as a standalone device or may be connected (e.g., networked) to other machines. In a networked deployment, the machine may operate in the capacity of a server machine or a client machine in a server-client network environment, or as a peer machine in a peer-to-peer (or distributed) network environment.

The structure of a computing machine described in FIG. 5 may correspond to any software, hardware, or combined components shown in FIGS. 1 and 2, including but not limited to, the client device 110, the computing server 130, and various engines, interfaces, terminals, and machines shown in FIG. 2. While FIG. 5 shows various hardware and software elements, each of the components described in FIGS. 1 and 2 may include additional or fewer elements.

By way of example, a computing machine may be a personal computer (PC), a tablet PC, a set-top box (STB), a personal digital assistant (PDA), a cellular telephone, a smartphone, a web appliance, a network router, an internet of things (IoT) device, a switch or bridge, or any machine capable of executing instructions 524 that specify actions to be taken by that machine. Further, while only a single machine is illustrated, the terms “machine” and “computer” may also be taken to include any collection of machines that individually or jointly execute instructions 524 to perform any one or more of the methodologies discussed herein.

The example computer system 500 includes one or more processors 502 such as a CPU (central processing unit), a GPU (graphics processing unit), a TPU (tensor processing unit), a DSP (digital signal processor), a system on a chip (SOC), a controller, a state equipment, an application-specific integrated circuit (ASIC), a field-programmable gate array (FPGA), or any combination of these. Parts of the computing system 500 may also include a memory 504 that stores computer code including instructions 524 that may cause the processors 502 to perform certain actions when the instructions are executed, directly or indirectly by the processors 502. Instructions may be any directions, commands, or orders that may be stored in different forms, such as equipment-readable instructions, programming instructions including source code, and other communication signals and orders. Instructions may be used in a general sense and are not limited to machine-readable codes. One or more steps in various processes described may be performed by passing through instructions to one or more multiply-accumulate (MAC) units of the processors.

One or more methods described herein improve the operation speed of the processor 502 and reduce the space required for the memory 504. For example, the database processing techniques and machine learning methods described herein reduce the complexity of the computation of the processors 502 by applying one or more novel techniques that simplify the steps in training, reaching convergence, and generating results of the processors 502. The algorithms described herein also reduce the size of the models and datasets to reduce the storage space requirement for memory 504.

The performance of certain operations may be distributed among more than one processor, not only residing within a single machine, but deployed across a number of machines. In some example embodiments, the one or more processors or processor-implemented modules may be located in a single geographic location (e.g., within a home environment, an office environment, or a server farm). In other example embodiments, one or more processors or processor-implemented modules may be distributed across a number of geographic locations. Even though the specification or the claims may refer to some processes to be performed by a processor, this may be construed to include a joint operation of multiple distributed processors. In some embodiments, a computer-readable medium comprises one or more computer-readable media that, individually, together, or distributively, comprise instructions that, when executed by one or more processors, cause the one or more processors to perform, individually, together, or distributively, the steps of the instructions stored on the one or more computer-readable media. Similarly, a processor comprises one or more processors or processing units that, individually, together, or distributively, perform the steps of instructions stored on a computer-readable medium. In various embodiments, the discussion of one or more processors that carry out a process with multiple steps does not require any one of the processors to carry out all of the steps. For example, a processor A can carry out step A, a processor B can carry out step B using, for example, the result from the processor A, and a processor C can carry out step C, etc. The processors may work cooperatively in this type of situation such as in multiple processors of a system in a chip, in Cloud computing, or in distributed computing.

The computer system 500 may include a main memory 504, and a static memory 506, which are configured to communicate with each other via a bus 508. The computer system 500 may further include a graphics display unit 510 (e.g., a plasma display panel (PDP), a liquid crystal display (LCD), a projector, or a cathode ray tube (CRT)). The graphics display unit 510, controlled by the processor 502, displays a graphical user interface (GUI) to display one or more results and data generated by the processes described herein. The computer system 500 may also include an alphanumeric input device 512 (e.g., a keyboard), a cursor control device 514 (e.g., a mouse, a trackball, a joystick, a motion sensor, or other pointing instruments), a storage unit 516 (a hard drive, a solid-state drive, a hybrid drive, a memory disk, etc.), a signal generation device 518 (e.g., a speaker), and a network interface device 520, which also are configured to communicate via the bus 508.

The storage unit 516 includes a computer-readable medium 522 on which is stored instructions 524 embodying any one or more of the methodologies or functions described herein. The instructions 524 may also reside, completely or at least partially, within the main memory 504 or within the processor 502 (e.g., within a processor's cache memory) during execution thereof by the computer system 500, the main memory 504 and the processor 502 also constituting computer-readable media. The instructions 524 may be transmitted or received over a network 526 via the network interface device 520.

While computer-readable medium 522 is shown in an example embodiment to be a single medium, the term “computer-readable medium” should be taken to include a single medium or multiple media (e.g., a centralized or distributed database, or associated caches and servers) able to store instructions (e.g., instructions 524). The computer-readable medium may include any medium that is capable of storing instructions (e.g., instructions 524) for execution by the processors (e.g., processors 502) and that cause the processors to perform any one or more of the methodologies disclosed herein. The computer-readable medium may include, but not be limited to, data repositories in the form of solid-state memories, optical media, and magnetic media. The computer-readable medium does not include a transitory medium such as a propagating signal or a carrier wave.

### Additional Considerations

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Any feature mentioned in one claim category, e.g. method, can be claimed in another claim category, e.g. computer program product, system, or storage medium, as well. The dependencies or references in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject matter may include not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein may be claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcodes, or the like. Furthermore, it has also proven convenient at times, to refer to these arrangements of operations as engines, without loss of generality. The described operations and their associated engines may be embodied in software, firmware, hardware, or any combinations thereof.

Any of the steps, operations, or processes described herein may be performed or implemented with one or more hardware or software engines, alone or in combination with other devices. In some embodiments, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure may describe a process that includes multiple steps sequentially with arrows present in a flowchart, the steps in the process do not need to be performed in the specific order claimed or described in the disclosure. Some steps may be performed before others even though the other steps are claimed or described first in this disclosure. Likewise, any use of (i), (ii), (iii), etc., or (a), (b), (c), etc. in the specification or in the claims, unless specified, is used to better enumerate items or steps and also does not mandate a particular order.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not imply that all members are associated with an element A. Instead, the term “each” only implies that a member (of some of the members), in a singular form, is associated with an element A. In claims, the use of a singular form of a noun may imply at least one element even though a plural form is not used.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

The following applications are incorporated by reference in their entirety for all purposes: (1) U.S. Pat. No. 10,679,729, entitled “Haplotype Phasing Models,” granted on Jun. 9, 2020, (2) U.S. Pat. No. 10,720,229, entitled “Reducing Error in Predicted Genetic Relationships,” granted on Jul. 21, 2020, (3) U.S. Pat. No. 10,558,930, entitled “Local Genetic Ethnicity Determination System,” granted on Feb. 11, 2020, (4) U.S. Pat. No. 10,114,922, entitled “Identifying Ancestral Relationships Using a Continuous Stream of Input,” granted on Oct. 30, 2018, (5) U.S. Pat. No. 11,429,615, entitled “Linking Individual Datasets to a Database,” granted on Aug. 30, 2022, (6) U.S. Pat. No. 10,692,587, entitled “Global Ancestry Determination System,” granted on Jun. 23, 2020, and (7) U.S. Patent Application Publication No. US 2021/0034647, entitled “Clustering of Matched Segments to Determine Linkage of Dataset in a Database,” published on Feb. 4, 2021.

1. A computer-implemented method, comprising:
receiving a genomic dataset of a target domestic companion animal that belongs to a first owner, the first owner being a user of an online system;
accessing genomic datasets of reference panel animals, wherein the reference panel animals are organized by breeds, wherein organizing the reference panel animals by breeds comprises:
dividing the genomic datasets of the reference panel animals into a plurality of genomic windows, each genomic window comprising a plurality of single nucleotide polymorphisms (SNPs),
classifying the genomic datasets window by window into a plurality of breeds based on values of the SNPs in each genomic window,
determining confidence levels of the reference panel animals being assigned to the breeds, and
filtering the reference panel animals based on a confidence level threshold;
dividing the genomic dataset of the target domestic companion animal into the plurality of genomic windows;
determining a breed composition of the target domestic companion animal by comparing the genomic dataset of the target domestic companion animal that is divided into the plurality of windows to the genomic datasets of the reference panel animals to identify one or more breeds of the target domestic companion animal based on comparisons in the plurality of windows;
identifying a plurality of matched domestic companion animals based on the one or more breeds of the target domestic companion animal, wherein identifying the plurality of matched domestic companion animals comprises matching the one or more breeds of the target domestic companion animal to breeds of the matched domestic companion animals;
filtering the matched domestic companion animals based on geographical proximity between locations of the target domestic companion animal and the matched domestic companion animals; and
causing to display, at a graphical user interface, a filtered matched domestic companion animal to the first owner of the target domestic companion animal to indicate that the filtered matched domestic companion animal and the target domestic companion animal are a potential social match.

2. The computer-implemented method of claim 1, wherein the reference panel animals are generated by:
organizing a genetic network formed of the reference panel animals; and
determining one or more breeds from the genetic network, wherein each breed comprises at least five distinct samples of a purebred animal of the breed, and wherein the at least five distinct samples are selected from the reference panel animals.

3. The computer-implemented method of claim 2, wherein
determining the one or more breeds from the reference panel animals comprises:
determining each of the breeds from the genomic datasets with a confidence in breed identification of at least a threshold.

4. The computer-implemented method of claim 1, further comprising:
determining genetic matched segments between the target domestic companion animal and other domestic companion animals, wherein the matched segments comprise identity-by-descent (IBD) segments shared between the target domestic companion animal and the other domestic companion animals; and
reporting that the other domestic companion animals are potentially familial match to the target domestic companion animal.

5. The computer-implemented method of claim 1, wherein identifying the plurality of matched domestic companion animals further comprises:
determining the matched domestic companion animals based on a breed type of the target domestic companion animal.

6. The computer-implemented method of claim 5, wherein determining the matched domestic companion animals based on the breed type of the target domestic companion animal comprises:
grouping a set of breeds into the breed type; and
determining whether a matched domestic companion animal and the target domestic companion animal share the same breed type.

7. The computer-implemented method of claim 6, wherein grouping the set of breeds into the breed type comprises:
determining whether the set of breeds are compatible based on energy levels of the breeds in the set.

8. The computer-implemented method of claim 6, wherein grouping the set of breeds into the breed type comprises:
determining whether the set of breeds are compatible based on sizes, weights or colors of the breeds in the set.

9. The computer-implemented method of claim 1, wherein causing to display, at the graphical user interface, the filtered matched domestic companion animal to the first owner of the target domestic companion animal comprises:
causing to display a messaging feature that enables communication between the first owner of the target domestic companion animal and the other owners of the matched domestic companion animals.

10. A system comprising:
one or more processors; and
memory configured to store instructions, wherein the instructions, when executed by the one or more processors, cause the one or more processors to perform steps comprising:
receiving a genomic dataset of a target domestic companion animal that belongs to a first owner, the first owner being a user of an online system;
accessing genomic datasets of reference panel animals,
wherein the reference panel animals are organized by breeds,
wherein organizing the reference panel animals by breeds comprises:
dividing the genomic datasets of the reference panel animals into a plurality of genomic windows, each genomic window comprising a plurality of single nucleotide polymorphisms (SNPs),
classifying the genomic datasets window by window into a plurality of breeds based on values of the SNPs in each genomic window,
determining confidence levels of the reference panel animals being assigned to the breeds, and
filtering the reference panel animals based on a confidence level threshold;
dividing the genomic dataset of the target domestic companion animal into the plurality of genomic windows;
determining a breed composition of the target domestic companion animal by comparing the genomic dataset of the target domestic companion animal that is divided into the plurality of windows to the genomic datasets of the reference panel animals to identify one or more breeds of the target domestic companion animal based on comparisons in the plurality of windows;
identifying a plurality of matched domestic companion animals based on the one or more breeds of the target domestic companion animal, wherein identifying the plurality of matched domestic companion animals comprises matching the one or more breeds of the target domestic companion animal to breeds of the matched domestic companion animals;
filtering the matched domestic companion animals based on geographical proximity between locations of the target domestic companion animal and the matched domestic companion animals; and
causing to display, at a graphical user interface, a filtered matched domestic companion animal to the first owner of the target domestic companion animal to indicate that the filtered matched domestic companion animal and the target domestic companion animal are a potential social match.

11. The system of claim 10, wherein the reference panel animals are generated by:
organizing a genetic network formed of the reference panel animals; and
determining one or more breeds from the genetic network, wherein each breed comprises at least five distinct samples of a purebred animal of the breed, and wherein the at least five distinct samples are selected from the reference panel animals.

12. The system of claim 11, wherein determining the one or more breeds from the reference panel animals comprises:
determining each of the breeds from the genomic datasets with a confidence in breed identification of at least a threshold.

13. The system of claim 10, wherein the steps further comprise:
determining genetic matched segments between the target domestic companion animal and other domestic companion animals, wherein the matched segments comprise identity-by-descent (IBD) segments shared between the target domestic companion animal and the other domestic companion animals; and
reporting that the other domestic companion animals are potentially familial match to the target domestic companion animal.

14. The system of claim 10, wherein identifying the plurality of matched domestic companion animals further comprises:
determining the matched domestic companion animals based on a breed type of the target domestic companion animal.

15. The system of claim 14, wherein determining the matched domestic companion animals based on the breed type of the target domestic companion animal comprises:
grouping a set of breeds into the breed type; and
determining whether a matched domestic companion animal and the target domestic companion animal share the same breed type.

16. The system of claim 15, wherein grouping the set of breeds into the breed type comprises:
determining whether the set of breeds are compatible based on energy levels of the breeds in the set.

17. The system of claim 15, wherein grouping the set of breeds into the breed type comprises:
determining whether the set of breeds are compatible based on sizes, weights or colors of the breeds in the set.

18. The system of claim 10, wherein causing to display, at the graphical user interface, the filtered matched domestic companion animal to the first owner of the target domestic companion animal comprises:
causing to display a messaging feature that enables communication between the first owner of the target domestic companion animal and the other owners of the matched domestic companion animals.

19. A non-transitory computer readable medium for storing computer code comprising instructions, when executed by one or more computer processors, causing one or more computer processors to perform steps comprising:
receiving a genomic dataset of a target domestic companion animal that belongs to a first owner, the first owner being a user of an online system;
accessing genomic datasets of reference panel animals, wherein the reference panel animals are organized by breeds, wherein organizing the reference panel animals by breeds comprises:
dividing the genomic datasets of the reference panel animals into a plurality of genomic windows, each genomic window comprising a plurality of single nucleotide polymorphisms (SNPs),
classifying the genomic datasets window by window into a plurality of breeds based on values of the SNPs in each genomic window,
determining confidence levels of the reference panel animals being assigned to the breeds, and
filtering the reference panel animals based on a confidence level threshold;
dividing the genomic dataset of the target domestic companion animal into the plurality of genomic windows;
determining a breed composition of the target domestic companion animal by comparing the genomic dataset of the target domestic companion animal that is divided into the plurality of windows to the genomic datasets of the reference panel animals to identify one or more breeds of the target domestic companion animal based on comparisons in the plurality of windows;
identifying a plurality of matched domestic companion animals based on the one or more breeds of the target domestic companion animal, wherein identifying the plurality of matched domestic companion animals comprises matching the one or more breeds of the target domestic companion animal to breeds of the matched domestic companion animals;
filtering the matched domestic companion animals based on geographical proximity between locations of the target domestic companion animal and the matched domestic companion animals; and
causing to display, at a graphical user interface, a filtered matched domestic companion animal to the first owner of the target domestic companion animal to indicate that the filtered matched domestic companion animal and the target domestic companion animal are a potential

20. The non-transitory computer readable medium of claim 19, wherein the reference panel animals are generated by:
organizing a genetic network formed of the reference panel animals; and
determining one or more breeds from the genetic network, wherein each breed comprises at least five distinct samples of a purebred animal of the breed, and wherein the at least five distinct samples are selected from the reference panel animals.
